(19) United States
(12) Patent Application Publication
(10) Pub. No.: US 2026/0094710 A1
(43) Pub. Date: Apr. 2, 2026

(54) RESPIRATORY EVALUATION AND MONITORING SYSTEM AND METHOD

(71) Applicants: Hadassa HARTMAN, Mizpei Ilan (IL); Israel AMIRAV, Kiryat Ono (IL); Yossi AVNI, Caesarea (IL); Adi SHARVAS, Herzliya (IL)

(72) Inventors: Hadassa HARTMAN, Mizpei Ilan (IL); Israel AMIRAV, Kiryat Ono (IL); Yossi AVNI, Caesarea (IL); Adi SHARVAS, Herzliya (IL)

(21) Appl. No.: 19/111,436

(22) PCT Filed: Sep. 14, 2023
(86) PCT No.: PCT/IB2023/059150

§ 371 Date: Mar. 13, 2025

Related U.S. Application Data
(60) Provisional application No. 63/406,748, filed on Sep. 15, 2022.

(51) Int. Cl. G16H 50/20; G16H 40/63

(52) U.S. Cl. CPC G16H 50/20; G16H 40/63

(57) ABSTRACT

A system and method of monitoring tidal breath even in infants, including: passively capturing video imagery of a side-view of a monitored subject during tidal breathing, wherein an imaging device capturing the video is not physical contact with the subject body; analyzing the video imagery to define a region of interest (ROI); analyzing the ROI to determine Thoraco-Abdominal Asynchrony (TAA).

### FIELD OF THE INVENTION

The present invention relates to a method and system for monitoring respiration, especially in children, and, more specifically, to using computer vision to assess the monitored parameters, even without physical contact with the monitored subject.

### BACKGROUND OF THE INVENTION

The NIH defines pulmonary disease as a type of disease that affects the lungs and other parts of the respiratory system. Pulmonary diseases may have many causes including infection, allergies, and environmental factors such as NO2, tobacco smoke, radon, asbestos, Ethylene Oxide, or other forms of air pollution. Pulmonary diseases may include broncho-pulmonary diseases of infants, bronchiolitis, asthma, chronic obstructive pulmonary disease (COPD), pulmonary fibrosis, pneumonia, and lung cancer. Also called lung disorder and respiratory disease.

Pulmonary diseases are the most frequent chronic condition causing hospitalizations in children and second most common in adults. Diagnosis, monitoring and managing pulmonary diseases is a high priority for patients, caregivers, and health suppliers.

There are multiple additional medical conditions that require pulmonary status evaluations and monitoring. Some of those conditions include patients in NICU, PICU, CF patients, spinal muscular atrophy (SMA), patients on mechanical ventilation, pneumonia and more. These patients would also benefit from pulmonary evaluation and monitoring both at the medical center and upon returning home.

Key stages in successfully handling the pulmonary cycle of care of these diseases include evaluation of the pulmonary status, monitoring response to prescribed treatment, and detecting an impending exacerbation in order to adjust or prescribe additional medication before the full onset of the exacerbation. If any of the stages is not effective, an exacerbation may deteriorate, requiring additional medications, clinic visits, emergency department (ED) visits, and possibly even hospitalization and death. Many of these stages are currently lacking appropriate tools, especially tools adapted for the younger population, both in the clinical setting and for home use. The proposed system addresses two elements in the cycle of care that are lacking tools: diagnostic tools that are adapted to the young population and/or can provide objective measurable medical data, and tools for long-term monitoring to assist with the early detection of exacerbations.

Lung function tests (LFT) are used to diagnose and monitor progression of illness, and abnormal functionality can be an important marker of serious illness. LFTs are most commonly performed using spirometers-a device that requires patients to cooperate and perform forced inhalations and exhalations into the device. These tests require a minimal degree of patient cooperation and forced expiration maneuvers but are not possible for the youngest population (0-5 years old). Alternative techniques developed for young patients consist of various measures obtained from tidal breaths. These have provided important clinical respiratory data. All current measurements of tidal breathing involve various devices, however, such methods are costly, and require access to specialized medical centers, making them unsuitable for frequent testing that could otherwise provide early diagnosis or continuous tracking of disease progression. Tidal breaths have been evaluated, for example, using devices such as respiratory inductive plethysmography (RIP) bands or piezoelectric belts that use transducers to convert thoraco-abdominal (TA) movement into changes in voltage. Although this method is appropriate for young children, it is not widely used due to the cumbersome and expensive setup. Due to the lack of tools available for diagnosis and disease management in young children, diagnosis and disease management are mainly based on parental descriptions and subjective non-measurable evaluations. There is a strong need to detect asthma in this age group as early detection has implications for future respiratory health. Technologies are currently being developed to address this gap.

Structured light plethysmography (SLP) is a new technology that has emerged in recent years and is suitable for young children, as it is a no-contact solution and requires minimal cooperation. SLP technology involves monitoring the thoracic-abdominal (TA) wall displacement using a depth camera. This technology is adapted to most of the younger population and has been tested on children 2 years old and above and has proven the potential for the use of TA as a marker for pulmonary status in children with Asthma (https://pubmed.ncbi.nlm.nih.gov/29932498/). Although this is a step forward, SLP technology is not adapted for home use and requires the child's cooperation for the lengthy recordings.

### SUMMARY OF THE INVENTION

According to the present invention, there is provided a method of monitoring tidal breath even in infants, including: passively capturing video imagery of a monitored subject during tidal breathing, wherein an imaging device capturing the video is not in physical contact with the subject body; analyzing the video imagery to define a region of interest (ROI); analyzing the ROI to determine a Thoraco-Abdominal Asynchrony (TAA).

According to further features in example embodiments of the invention, the ROI is determined by running at least a portion of the video imagery through a trained machine learning (ML) model.

According to still further features in the described preferred embodiments, the TAA is determined by running at least a portion of the video imagery through a trained machine learning (ML) model. According to further features, the ML model provides a diagnosis based on the video imagery. According to further features, the ML model determines indicators of one or more medical conditions based on the video imagery. According to further features, the video imagery is added to a dataset upon which the ML model is trained.

According to further features, the method further includes receiving motion data from motion sensors in physical contact with the monitored subject. According to further features, the method further includes receiving audio data from audio sensors in physical contact with the monitored subject. According to further features, the motion sensors, the audio sensors, or both are disposed on a patch that is adapted to be positioned in physical contact with the monitored subject.

According to further features, the video is captured using an integrated camera of a portable computing device (PCD). According to further features, the PCD has a diagnostic program installed thereon, the diagnostic program configured to analyze the captured video. According to further features, the PCD has a communications application installed thereon, wherein the communications application is configured to send or stream the captured video to an off-site computing device, wherein the off-site computing device has a diagnostic program installed thereon, the diagnostic program configured to analyze the captured video.

According to further features, the method further includes providing an alert when distressed breathing is detected.

According to further features, parameters of pulmonary status data are determined by running at least a portion of the video imagery through a trained machine learning (ML) model, the pulmonary status data including: inspiratory time (tI), expiratory time (tE), time to peak tidal expiratory flow/expiratory time (tPTEF/tE), time to peak tidal inspiratory flow/inspiratory time (tPTIF/tI), ratio of inspiratory to expiratory flow at 50% tidal volume (IE50), Relative thoracic contribution, and TAA.

According to further features, the method further includes providing an alert when monitored pulmonary status data parameters match deterioration patterns identified through the ML model. According to further features, the method further includes providing an alert when monitored pulmonary status data parameters show deterioration when compared to previously monitored pulmonary status data parameters for the monitored subject. According to further features, the method further includes providing an alert when monitored pulmonary status data parameters indicate asthmatic exacerbation triggers.

### BRIEF DESCRIPTION OF THE DRAWINGS

Various embodiments are herein described, by way of example only, with reference to the accompanying drawings, wherein:

FIG. 1 is an example Full-Stack architecture of an embodiment of the instant system;

FIGS. 2(a)-2(c) are image processing techniques for detecting the different ROI, using both full and occluded images of the body;

FIG. 3 is a sensor patch as used in embodiments of the invention;

FIG. 4 is an example dashboard/analysis tool 150 for analyzing offline or real-time video remotely;

FIG. 5 is a tidal breathing analysis inspection tool 152;

FIG. 6 is a view of comparative graphs 160 of chest and abdominal movement;

FIG. 7 is a tool 154 for analyzing real-time video;

FIG. 8 is a diagram of the main actions and flow of a symptom exacerbation prediction process 800;

FIG. 9 is a flow diagram of a process 900 of captured imagery analysis;

FIG. 10 is an RNN deep learning model architecture;

FIG. 11 is a calculated phase shift displayed on a screen 1100 in the case of a correctly held smartphone;

FIG. 12 is a calculated phase shift displayed on a screen 1200 in the case of an incorrectly held smartphone.

### DESCRIPTION OF THE PREFERRED EMBODIMENTS

With the advancement of telehealth, there are new opportunities to improve remote diagnosis, monitoring, and guidance. Using a technology that is already embedded into everyday life, such as smartphones, allows for a cost-effective solution and for improved chances for patients' compliance. There is provided herein a camera-based, non-contact monitoring system that passively images a side-view of a monitored subject. As mentioned, the camera may even be a smartphone camera (or other digital camera or video recorder).

Current devices in the field that incorporate the use of cameras for monitoring Thoraco-Abdominal (TA) wall displacement are based on several available technologies. The available technologies utilize monitoring of RGB changes and/or use specialized depth cameras. By contrast, the instant system uses passively captured video imagery. The video imagery is processed using computer vision. In embodiments, machine learning (ML) models are used to process the video imagery.

The terms Artificial Intelligence (AI), machine learning (ML), ML models and variations thereof are used interchangeably herein and are intended to be generic terms referring to any and all applicable methodologies under the wide umbrella of machine learning.

The currently available technologies do not automatically detect the region of interest to monitor and/or require long recordings, which require patient cooperation, and/or are sensitive to environmental interference, and/or are not adapted for home use, and/or require specific recording settings, and/or only provide the patients' current status and/or require mandatory additional accessories.

The technology presented herein incorporates the idea of non-contact monitoring of TA wall displacement and tidal breathing monitoring, with the option of adding an additional specialized “patch” with sensors for added accuracy. The System introduces improved technology for better accuracy and home-based usability and incorporates the monitoring of the novel tidal breathing parameter, namely Thoraco-Abdominal Asynchrony (TAA), also referred to as “phase shift” (the terms are used interchangeably herein).

In embodiments, the system utilizes a camera (e.g., a smartphone camera) alone to capture the video imagery. In other embodiments, the system utilizes both the camera and a specialized “patch” that is placed in contact with the monitored subject. The patch includes motion sensors disposed on the patch and configured to evaluate the tidal breathing status.

In embodiments, the system employs Artificial Intelligence (AI) and/or other machine learning (ML) models and methodologies to detect the region of interest (ROI) from at least a portion of the video imagery captured by the smartphone camera and/or an external camera. The ROI is detected, inter alia, by neutralizing surrounding interference. The system automatically selects the type of image analysis required based on recording indicators. Specialized software (SW) is employed to reconstruct breathing patterns from the video imagery, even in embodiments without contact sensors.

In embodiments that include motion sensors, the breathing pattern reconstruction can also be performed using the motion sensors. These motion sensors are placed at strategic locations on the subject. In addition, the video imagery of the TA movement is processed using computer vision, so that the motion sensor data and image data are processed to, inter alia, reconstruct the breathing patterns of the monitored subject. In some embodiments, AI/ML models are employed to perform or enhance the process of image processing. In embodiments, the AI then refines the collected data and provides the patient and the medical staff with concise, accurate, reliable, objective, and measurable pulmonary status data.

Pulmonary status data that can be derived using one or more of the methods discussed above (e.g., processing image data, processing image data and motion sensor data, AI-assisted processing of the motion and/or image data), includes, but is not limited to: respiratory rate, coughing monitoring, inspiratory time (tI), expiratory time (tE), tPTEF/tE (time to peak tidal expiratory flow/expiratory time), tPTIF/tI (time to peak tidal inspiratory flow/inspiratory time), IE50 (ratio of inspiratory to expiratory flow at 50% tidal volume), Relative thoracic contribution (%), and/or TAA (Thoraco-abdominal asynchrony-phase shift).

This last indicator, phase shift, is a novel concept in many respiratory conditions associated with respiratory distress and increased work of breathing. A common condition where respiratory distress is vital and can be missed is Asthma exacerbations. Monitoring changes in the synchronization of the chest and abdomen movements, as indicators of asynchrony between the movement of the chest and the diaphragm, is a valuable indicator for asthma exacerbation. The diaphragm contracts upon inhalation and expands upon exhalation. Changes in synchronization between the two muscle sets indicate changes in respiratory state. By monitoring the natural external movement of these internal organs, either through chest movements and abdominal movements (motion sensors) or by image processing of video clips (e.g., from a smartphone camera), the system provides measurable, objective, refined data. Slight changes in the synchronization of the two functions, thoracic periodic movement, and abdominal periodic movement, may be used as indications or markers of various pulmonary conditions.

In embodiments where the system includes the patch, additional indicators are monitored through the audio sensors disposed on the patch, including wheezing and/or heart rate and/or coughing. By placing the sensors at clinically significant locations, the sensors are more accurate for the younger pediatric population. These clinically significant locations include, but are not limited to: the armpit, lower lung location on the back, and/or top back. When synchronizing the audio indicators with TA movements, audio recordings that are not synced with the breathing pattern may be excluded, thus providing another layer of refining to the accumulated data and better accuracy.

The data provided by the system provides a measurable, objective standard for evaluating the pulmonary status of patients and provides an alternative to tools such as spirometers, which are not adapted for the home monitoring of young, non-compliant patients.

Long-term evaluation and early detection of exacerbation:

As mentioned above, another critical stage in the pulmonary cycle of care involves monitoring patients and identifying early signs of exacerbations.

Currently, there are several types of devices that assist with monitoring and treating exacerbations. The main types of devices that are available include,
1) An app (software application usually installed on a portable computing device such as a smartphone, tablet, or laptop) to assist with inhaled medication administration monitoring, coupled with a device that clips onto the inhaler. Some of the apps include input regarding air quality. These types of devices are not necessarily suitable for the younger population and are not continuous monitors of vital signs.
2) Wheezing detection devices to identify deterioration. These systems are not continuous and are used when an exacerbation is already evident and when the device is actively used by the caregiver. These systems are also based solely on audio input and mostly focus on a single type episodic monitoring and have not been sufficiently tested for effectiveness.
3) Baby monitors available are not asthma-specific and do not use a combination of sensors to assure accuracy. In addition, most of the monitors available are aimed at night/sleep monitoring and not for continuous daily monitoring.
4) Continuous respiratory and cardiac signals monitoring devices. These types of devices are mostly not compatible with the younger population and require patients' compliance with placing the patch on their bodies. All the continuous devices currently available do not assess tidal breathing or phase shift, but rather focus on the other markers such as respiratory rate, heart rate, wheezing and coughing. Incorporating this phase shift novel data into the calculation/analysis adds another layer of accuracy that is not available in any other device. These devices also incorporate limited input on triggers to assist with prediction and are not aimed at the youngest populations, who are most at risk of hospitalization and whose parents are at the initial stages of learning how to identify exacerbations and manage the disease.

The current camera-based technologies previously mentioned are focused on a single point of evaluation, and do not provide continuous monitoring solutions or identification of impending exacerbations.

Currently, there is no available technology that allows a combination of passive sensitive and accurate monitoring, trigger monitoring, and managing systems for chronic pediatric (ages 0-5y) pulmonary diseases. Various embodiments of the system can be used continuously, e.g., when using the patch connection and/or when placing the external camera at a strategic location and continuously recording. Such embodiments of the system continuously evaluate the patient. Additionally, or alternatively, embodiments of the system can be used semi-continuously by taking multiple readings with the external camera/smartphone camera.

The system's SW and/or AI allows for monitoring of the changes over time and for providing alerts upon detection of any breathing deterioration, based on detected indicators. When monitoring visually using the smartphone camera and/or the external camera and when comparing multiple readings over time, changes in the tidal breathing patterns, with particular reference to phase shift, can be easily detected. The latter is proposed to be indicative of respiratory status changes and the identification of the potential onset of an exacerbation.

Asthma exacerbations have many possible triggers, including but not limited to: certain viruses, weather changes, air pollution such as NO2, allergens, smoke, and/or more environmental conditions. By adding input regarding patient exposure to these potential triggers, yet another layer of confidence is added to the detection of exacerbations. Furthermore, by learning the specific patient's reactions to triggers over time, the system increases the accuracy by personalizing the alert thresholds. In embodiments, a patient has a database or dataset composed from the recorded data (including raw data, processed data, portions thereof, and/or any combination of the aforementioned). This dataset is distinct from, and may be incorporated in, a general dataset/database of recorded data from comparable subjects.

The SW logs the refined data, and every new reading is compared to previous readings and is evaluated for changes. The current reading is also compared to a general database compiled from the general population of both healthy and sick patients. The SW incorporates into the analysis previous reactions and/or identifies trends and/or exposure to triggers. Monitoring the novel tidal breathing indicators and the phase shift indicator adds additional accuracy and reliability to the diagnosis. Through real-time monitoring of airway, lung, and/or heart sounds, in embodiments of the system that use visual imagery in combination with the patch, an exacerbation may be detected at its initialization before it is visually evident. The database serves as a dataset upon which to train machine learning models.

Embodiments of the system combine one or more of the following components/technologies in various combinations: visual image processing, clinically and strategically placed audio sensors, and/or motion sensors. One or more of these components are processed, and the data (raw and/or processed) is analyzed in one or more of the following manners: comparison to the patient's previous readings, and/or comparison to a general population database. Furthermore, additional features can be taken into consideration when analyzing and processing the data, for example, incorporation of input about external stimuli, incorporation of the novel phase-shift indicator, and/or improved usability adapted to remote monitoring of the young non-compliant patient (passive, non-contact, and/or use of commonly found imaging devices such as a smartphone camera). The various combinations and constellations of technology and methods allow for one or more of: personalized disease management, identification of respiratory exacerbation, and alerts to the caregivers to provide immediate treatment to prevent deterioration, and ultimately to better decision making.

The principles and operation of systems and methods for monitoring tidal breath, even in infants, according to the present invention, may be better understood with reference to the drawings and the accompanying description. The invention is a combination of both hardware and software components. The invention is designed to provide respiratory data and to predict asthma attacks in any patient, and in particular, in children at the age of 0 to 5 years old. The instant solutions serve as surrogates for conventional Lung function tests (LFT), which are an integral part of diagnosing and monitoring many lung diseases. Conventional LFTs are generally not viable for the 0-5-year-old age range. This is one of the key factors in this innovative solution. For the younger population, ages 0-5, the biomarker of tidal breathing can be used when lung function tests would normally be used in the older population. Use of AI can monitor and sensitively evaluate status over time and detect changes and effectiveness of treatment. In addition, the system can be effective in reducing drug development, time, and cost.

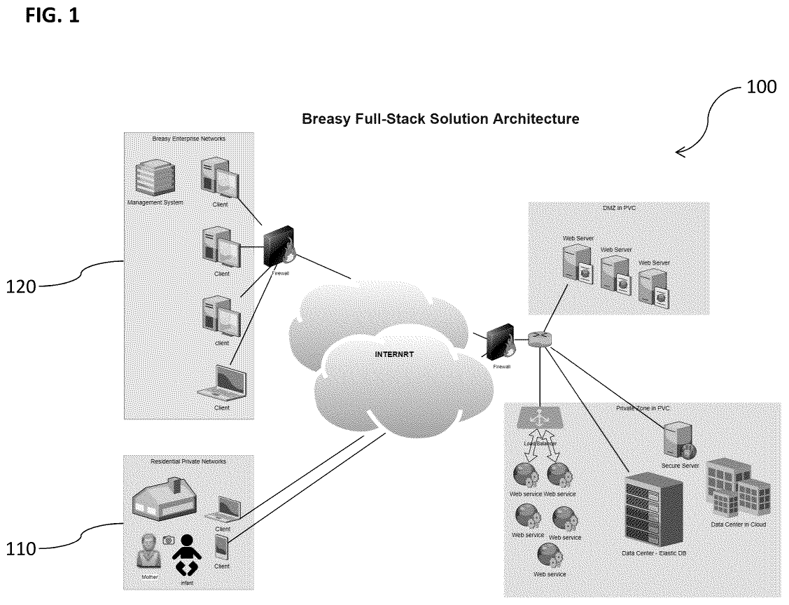

FIG. 1 illustrates an example Full-Stack architecture of an embodiment of the instant system. System 100 provides objective, refined pulmonary-related data to assist with disease diagnosis, enabling to prescribe the best treatment plan, monitor response to treatment, and/or alerting to an impending respiratory exacerbation for early treatment. In preferred embodiments, the system extracts the subject's breathing patterns without any bodily contact. The breathing signal is then fed to an artificial neural network (ANN—or other machine learning model) to assess pulmonary function in a passive manner, and there is zero effort needed from the patient and caregiver.

An exemplary implementation of the technology is a Full-Stack system that utilizes a remote end-point client-side application 110 running on smartphones, tablets, or PCs. The main components of the system include an application (SW) with AI-based algorithms running on the server-side service 120, which can communicate with a smartphone camera and/or an external camera. In some embodiments, service 120 is also in communication with a patch that includes, at least, a microcontroller and sensors.

In embodiments, the application (app) is installed on a smartphone device, a desktop device, a tablet computer, and/or electronic devices (the terms computing device, portable computing device [PCD], and variations thereof are used herein as a generic term that is intended to include all types of computing devices mentioned specifically herein, or that are known in the art). The app can communicate with an external camera (e.g., in a wired or wireless manner) and/or smartphone/tablet camera (especially if the app is installed on the same smartphone or tablet, collectively referred to herein as PCDs). In embodiments that include a patch (see below), the patch communicates with the computing device using secured and safe communication such as Bluetooth/Bluetooth Light Energy (BT/BLE) communication.

The main purpose is to enable a pure monitoring solution that can provide initial respiratory measurements without the need for any special hardware/device except for a smartphone/tablet with a camera. The potential use of a special sensor can provide improved accuracy and additional features such as the detection of wheezing, crackling, stridor, rhonchi, and whooping sounds. This high-pitched whistling noise can happen when patients are breathing in or out and is usually a sign that something is making the patient's airways narrow or keeping air from flowing through them.

In the case of smartphone/tablet use, the smartphone/tablet captures the video using its internal camera and sends it to the server-side service using Wi-Fi or cellular communication. The video file can be shared with another device and be sent using a different communication channel, such as ethernet networking. The app is used on a smartphone (the term “smartphone”, as used herein, is to be seen as including tablets as well as other portable computing devices that have an integrated camera) and can also directly interact with the smartphone's camera.

In embodiments, the app includes AI-based algorithms for processing the detected signals from the other system elements. Additionally, or alternatively, the app directs the collected signals to the cloud, processes the signals using advanced AI, and returns the refined data and calculated indicators as well as other information back to the system's app. The video analysis can be done locally or remotely using server-side services.

When using the smartphone camera and/or the external camera, the App can automatically detect regions of interest (ROI) using image processing. Detecting the ROI allows for improved monitoring of the chest and abdomen movements, which are later processed and refined to tidal breathing data by the App/server-side service. This detection involves non-contact detection and can be performed either in a synchronous manner (in real-time) or in an asynchronous manner.

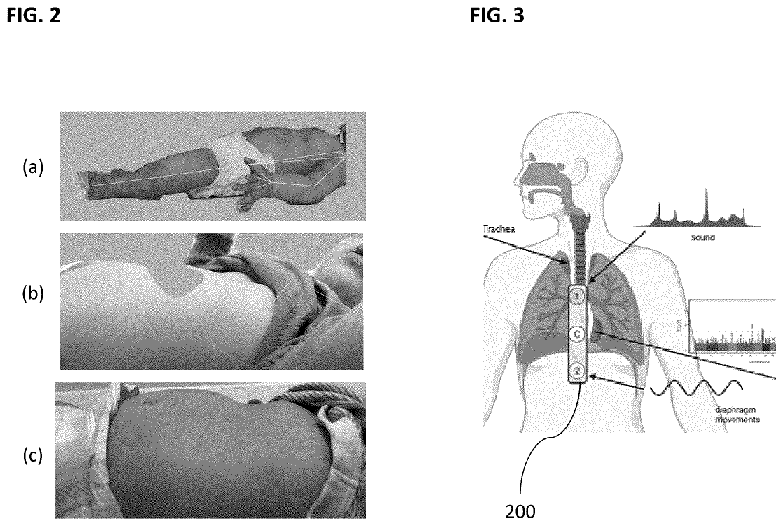

FIGS. 2(a)-(c) illustrate image processing techniques for detecting the different ROI, using both full and occluded images of the body. In embodiments, the ability to detect the ROI (Region of Interest), which includes both the chest and abdominal area, is based on AI models that detect body pose. In cases where most of the body is occluded or partially visible, image processing techniques can be used alternatively, or in addition to, the AI models. FIG. 2(a) depicts a side view of a full-body image. FIG. 2(b) depicts an occluded image of the ROI. FIG. 2(c) depicts another occluded image of a body. In embodiments, a side view of the monitored subject is imaged by the imaging device. In embodiments, the side view is between 0 and 60 degrees normal to a surface on which the monitored subject is resting. In embodiments, the side view is between 0 and 20 degrees normal to a surface on which the monitored subject is resting. In embodiments, the side view is between 0 and 15 degrees normal to a surface on which the monitored subject is resting.

In embodiments, the 2D video imagery is converted into a 3D model(s) for improved processing of the imagery of the monitored subject.

The capability to analyze a 2D video (optical camera) can be extended to 3D model capturing using the smartphone LiDAR camera which is available in any modern smartphone. This capability enables the system to provide a more reliable and efficient method to support capturing the scanned baby (monitored subject) from different angles without the limitation of capturing the baby body from side-view angle. In addition, it supports calculating volumetric measurements of the lungs during inhaling and exhaling movements. This can be very important for respiratory medical issues indicators.

In general, a LiDAR is used as a Light Detection and Ranging depth camera that utilizes remote sensing method which uses light in the form of a pulsed laser to measure ranges (variable distances) to the scanned object (a baby or a child). These light pulses are combined with other optical image data to generate precise, three-dimensional information about the shape of the scanned object's surface characteristics. The12canningg can be converted into points-cloud and/or 3D MESH formats.

Common point cloud formats include: LAS/LAZ, XYZ, and PLY. As described above, point clouds are collections of 3D points with additional, optional attributes. Point clouds, especially LAS/LAZ format, are more widely used in traditional geospatial workflows, and are supported in current versions of most GIS software. Common mesh formats include: GLTF/GLB, OBJ, FBX, STL, and USDZ. Mesh objects are 3D graphical models consisting of faces, edges, and vertices.

About the use of LiDAR camera in smartphones that are equipped with LiDAR camera and in particular in iPhones™. In 2020, Apple Inc. released the iPad Pro 2020™ and the iphone 12 Pro™ with build-in LiDAR sensors. LiDAR scanning data can be constructed using state-of-the-art Structure from Motion Multi-View Stereo (SfM MVS) point clouds. The LiDAR sensors create accurate high-resolution models of small objects with a side length >10 cm with an absolute accuracy of ±1 cm. Overall, the versatility in handling outweighs the range limitations, making the Apple LiDAR devices cost-effective alternatives to established techniques in remote sensing with possible fields of medical applications and in particular for analyzing respiratory medical disfunctions and issues.

The commonness of smartphones nowadays, together with advances in sensor technologies, opens new possibilities for scientific applications as well as low-cost solutions.

Another well-known way to render 2D images into a 3D model is by using a sequence of images. Images are one of the most commonly used data in recent deep learning models. Cameras are the sensors used to capture images. They take the points in the world and project them onto a 2D plane which we see as images. This transformation of 2D optical images (RGB and grayscale) to 3D model is usually divided into two parts: Extrinsic and Intrinsic. The extrinsic parameters of a camera depend on its location and orientation and have nothing to do with its internal parameters such as focal length, the field of view, etc. On the other hand, the intrinsic parameters of a camera depend on how it captures the images. Parameters such as focal length, aperture, field-of-view, resolution, etc. govern the intrinsic matrix of a camera model. These extrinsic and extrinsic parameters are transformation matrices that convert points from one coordinate system to the other.

The commonly used coordinate systems in Computer Vision are: World coordinate system (3D); Camera coordinate system (3D); Image coordinate system (2D); and Pixel coordinate system (2D).

The extrinsic matrix is a transformation matrix from the world coordinate system to the camera coordinate system, while the intrinsic matrix is a transformation matrix that converts points from the camera coordinate system to the pixel coordinate system.

In summary, the 3D object model can be constructed using the following transformations:
World-to-Camera: 3D-3D projection. Rotation, Scaling, Translation.
Camera-to-Image: 3D-2D projection. Loss of information. Depends on the camera model and its parameters (pinhole, f-theta, etc.).
Image-to-Pixel: 2D-2D projection. Continuous to discrete. Quantization and origin shift.

Camera Extrinsic Matrix (World-to-Camera): Converts points from world coordinate system to camera coordinate system. Depends on the position and orientation of the camera.

Camera Intrinsic Matrix (Camera-to-Image, Image-to-Pixel): Converts points from the camera coordinate system to the pixel coordinate system. Depends on camera properties (such as focal length, pixel dimensions, resolution, etc.).

FIG. 3 illustrates an example embodiment of a sensor patch as used in embodiments of the invention. In the case of using a sensor patch (special patch) 200, which is attached to the chest of the infant/child, the patch includes at least a microcontroller C and sensors 1 and 2. The patch may be a stand-alone patch, may be incorporated in a dedicated garment, and/or clipped on a garment. In example embodiments, two other acceleration and piezoelectric sensors 1 and 2 are attached to the chest and belly. Sensors 1 and 2 include an accelerator to measure both chest and abdominal movements in real time. In another example, or in addition to the features in the previous example, Sensor 1 can be, or includes, a tiny and sensitive microphone that can be located/attached at the optimized location to ensure minimum signal-to-noise ratio (SNR) and high-quality signals as much as possible. This sensor captures the lungs and heart sounds in real time. For example, Asthma produces a wheeze from the narrowing of the tracheobronchial tree from the diminished flow of air. This wheezing sound can be a significant sign of an oncoming asthma attack. During an asthma attack, there is a significant decrease in airflow exchange as the lungs hyperinflate. Sensor 2 can be, or includes, a tiny sensor such as MEMS (Micro Electronic Mechanical Systems) Acceleration Sensor (6 axes), PVDF sensor, or tiny gyroscope, which can be located/attached at the optimized location to ensure minimum SNR and high-quality signals as much as possible. This sensor can measure Diaphragm movements in real time.

The microcontroller processes signals from sensors placed at clinically strategic locations on the body. The patch may include one or more additional sensors, such as, one or more mini-microphones (to detect respiratory, heart sounds, and/or coughing), and/or a motion sensor to collect information about chest and abdomen movement. The microcontroller processes the captured signals from the sensors, synchronizes, labels, and transmits (e.g., via a secured communication channel) the information to the dedicated app and/or to the server-side service. The patch may be attached to the infant's chest either directly on the skin or indirectly, not touching the skin of the infant.

In some embodiments, in addition to the system elements, the app collects additional information about exacerbation triggers, such as, but not limited to, air pollution, weather forecast from online services, and/or additional condition-related manual input from a caregiver regarding allergy signs (nasal condition, skin rash, etc.) and patient background information.

All the data is processed, cleaned, integrated, and compared to a compiled general population database and general identified patterns and to the patient's previous baseline readings (personal database) to determine the current respiratory status and any changes in the patient's respiratory condition. The multitude of data collected when continuously using the system allows for advanced data mining, personalized treatment, trend learning, and extrapolation. The overall data is integrated and processed in multi-dimensional analysis to provide ongoing monitoring and even accurate predictions on oncoming distress, such as asthma attacks. The analysis and prediction are based on learning the patient's individual signals and patterns. In general, the AI/ML model is trained based on at least N number of patients (50% healthy and the other 50% suffering from Asthma; the training set consists of negative and positive signals/data). This baseline-trained model, using self-adaptive capabilities, learns each patient's breathing patterns regarding the trained baseline model, and the model is updated and automatically customized to the monitored patient. It means that for each patient, there is an adoption time where the model learns his/her individual patterns. Over time, the system will classify a number of breathing profiles/groups, enabling the overall analysis and prediction to be more accurate with more time and new patients. That is to say that all patients' patterns (healthy, in distress, after triggers, etc.) will eventually be compared both to their own previously monitored and stored patterns as well as to general (comparable) patterns and profiles identified from general databases.

The SW detects a decline in the respiratory and clinical data readings which are compared to the patient's baseline and “normal” patterns. The SW detects patterns that are similar to previous user's patterns associated with pre-exacerbation or similar to general public patterns identified through machine learning and accumulated databases. When used over time, the system can identify an exacerbation and alert the relevant caregivers to take appropriate and relevant steps to allow for early treatment and/or prevent deterioration, monitor the response to treatment and/or assist with diagnosis and communication with medical staff. When the system detects or predicts an oncoming attack, such as an asthma attack, the smartphone can immediately generate alerts, SMS, phone calls, iMessages™ to the patient and its caregivers, even before the attack occurs, supporting a preventive action to be executed at the right time and preventing, or at least minimizing the impact of an oncoming attack.

The history of patient's monitoring data can be used for medical inspection and research by the medical team and researchers. It may contain drugs and treatment effects along with the major factors that cause asthma attacks with timing and environmental factors.

The instant system is non-invasive, inexpensive, and provides an efficient solution for pulmonary monitoring, diagnosis, and follow-up for the younger population (ages 0-5). Using the instant system will reduce the burden on the medical system (clinic visits and hospitalizations), improve pulmonary condition diagnosis and treatment management, as well as improving the quality of life of both the patient and their caregivers.

The system introduces the capability of monitoring a biomarker called Thoraco-Abdominal Asynchrony (TAA or phase shift). This clinical biomarker, explained elsewhere herein, is a measurement that indicates increased respiratory distress. Unfortunately, measuring this feature was previously not easily accessible for the younger population (ages 0-5). The present invention now enables this measurement to be calculated remotely even in the young population of 0-3 or 0-5 years old.

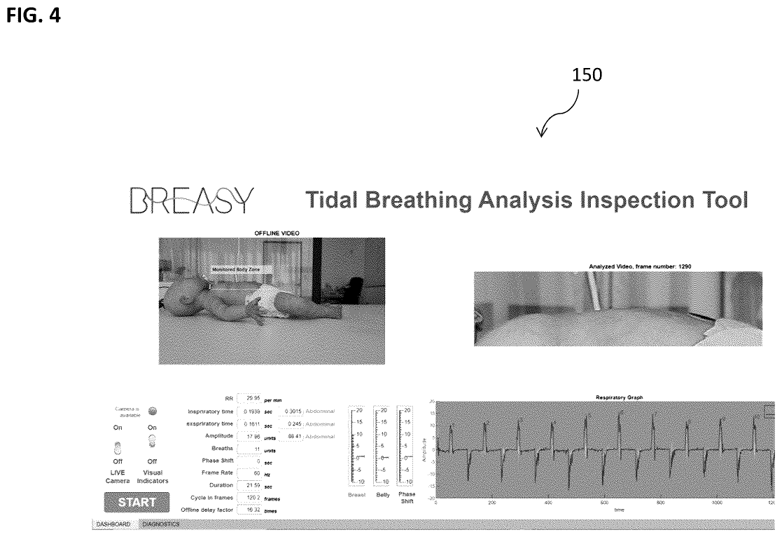

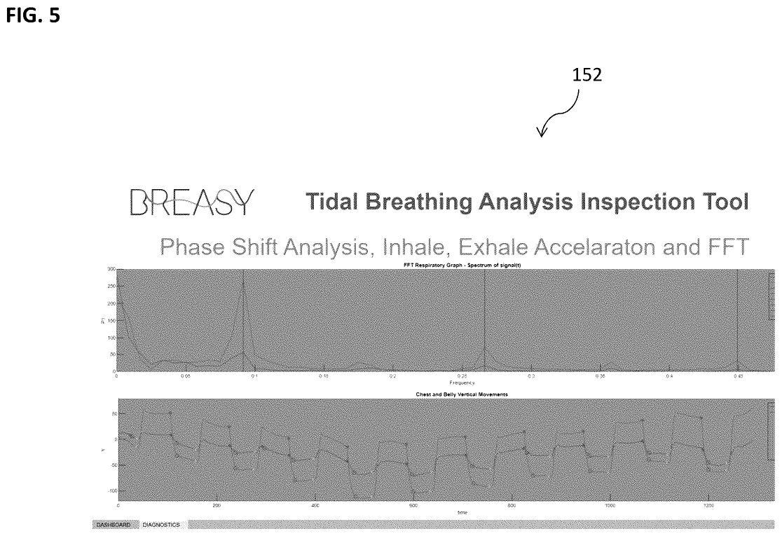

FIGS. 4-7 demonstrate the analysis findings regarding early diagnosis of respiratory health issues in infants. FIG. 4 illustrates an example dashboard/analysis tool 150 for analyzing offline or real-time video remotely. The results can be accessed by the physician remotely.

FIG. 5 illustrates a tidal breathing analysis inspection tool 152. The inspection tool can be used for the analysis of phase changes between chest and abdomen movements that may indicate respiratory health problems.

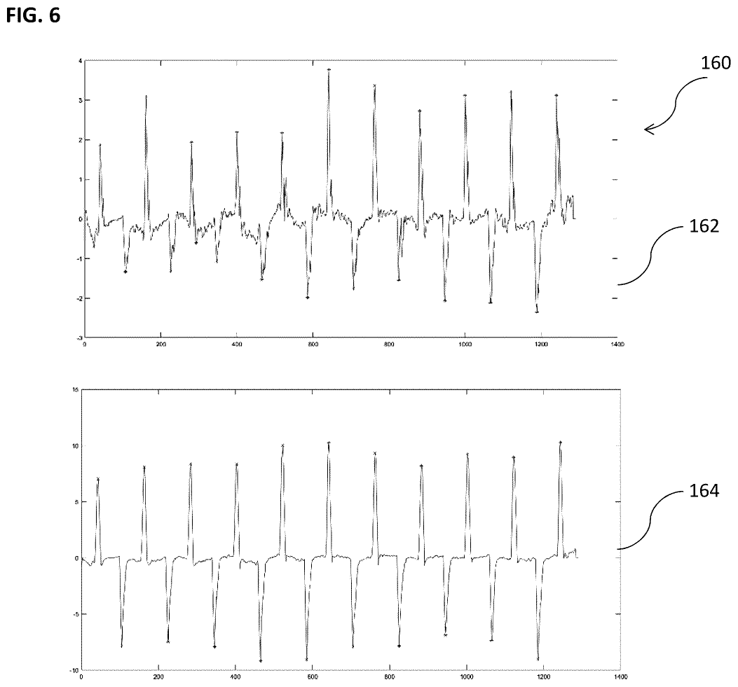

FIG. 6 illustrates comparative graphs 160 of chest and abdominal movement. The graphs depict an analysis of chest movements (on top graph 162) vs. abdominal movements (on bottom graph 164).

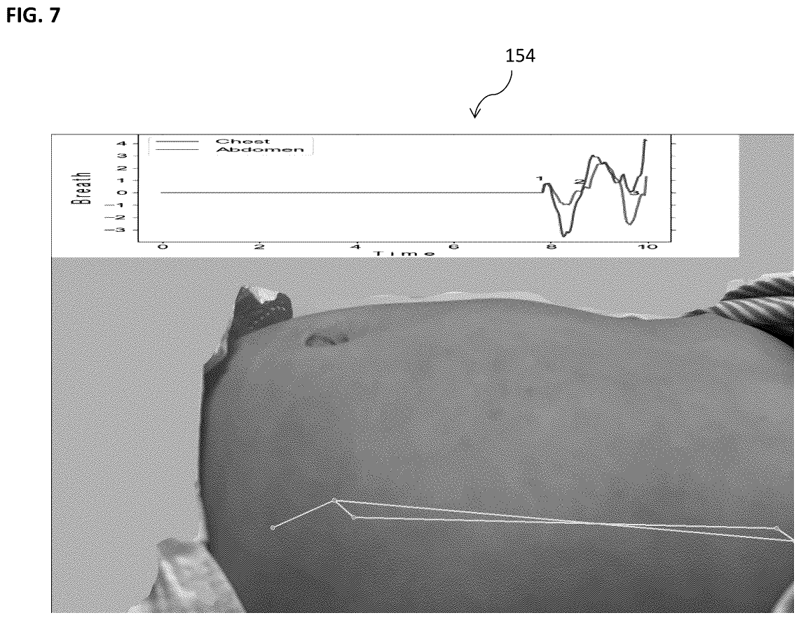

FIG. 7 depicts a tool 154 for analyzing real-time video (online mode) at the physician's smartphone, tablet, PC, etc. The SW tool displays the identification of the ROI and the presented patterns identified from the monitored chest and abdomen.

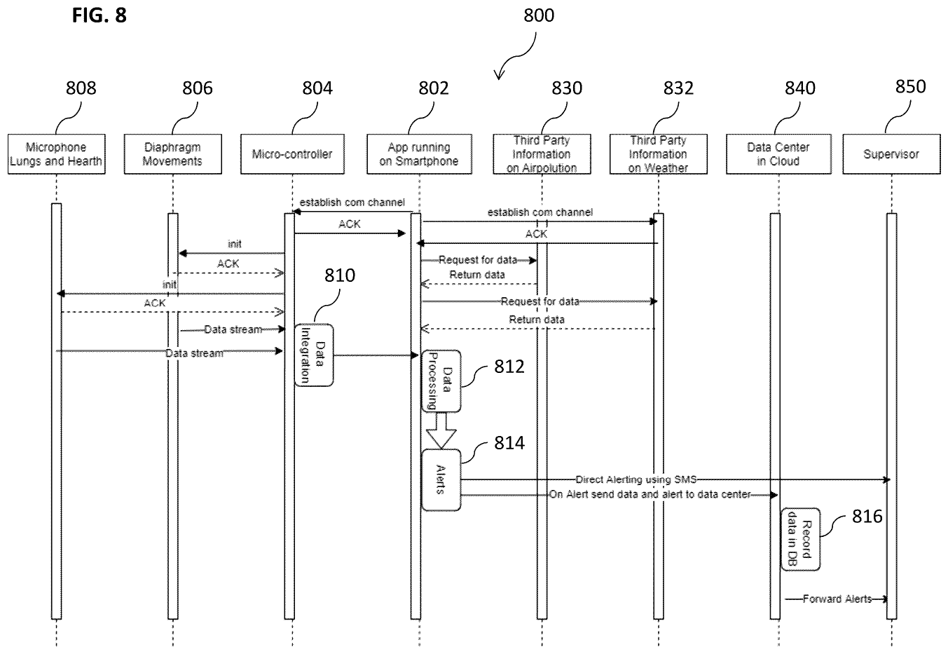

FIG. 8 describes the main actions and details a flow diagram of a symptom exacerbation prediction process 800. In Block 802, the App is installed and running on a smartphone. The App establishes secure communication channels with the data sources. In one embodiment, the data source is the integrated/internal camera of the computing device or portable computing device. In embodiments, the sensors further include installed sensors via microcontroller and third-party information services such as domestic air pollution centers 830 and domestic weather information service providers 832. Communication channels with the data center 840 and the caretakers 850 are established before sending and/or communicating with them.

In embodiments with a patch 200, once a communication channel with the attached microcontroller (sensor C), block 804, has been established, an initialization process is initiated with both the camera and sensors, (sensor 1, block 806, the special microphone, which samples sound from the lungs and heart area, and sensor 2, block 808, the MEMS Acceleration Sensor, which samples the diaphragm vertical movements regarding the body surface). Upon a successful initialization process, data from both camera and sensors, 1 and 2, are synced and sent to the microcontroller, sensor C. In embodiments without the patch, only permissions and, in some cases, a calibration process is necessary to sync between the app and the integrated camera.

At block 810, the data is streamed to the microcontroller, sensor c, and then cleaned/filtered from any irrelevant noise frequency or Gaussian noise. After cleaning the data, both data channels are integrated and sent to the running App (on the smartphone) via BT/BLE (Bluetooth communication channel).

At block 812 the running App, preprocesses the incoming streamed data (from sensor c), syncs it with other third-party data, normalizes and data pads it, and then uses features extraction data processing and generates it as n-dimensional time-series. These features are extracted in a similar procedure/protocol executed during the model/analyzer training process.

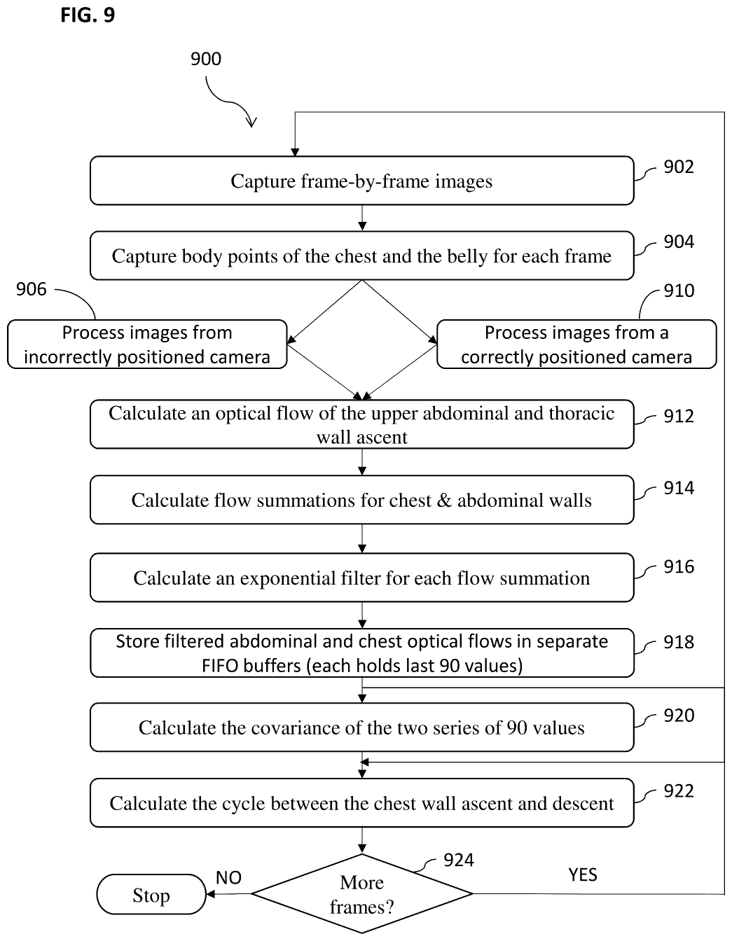

The App analyzes the breathing video imagery. See process 900 in FIG. 9.

Pre-processed time-series data is analyzed using a Long short-term memory (LSTM) network machine learning model. An LSTM network is a recurrent neural network (RNN), aimed to deal with the vanishing gradient problem present in traditional RNNs. Synced sound signatures/patterns with Asthma (or other pulmonary-related ailments) risk factors and the diaphragm movements are analyzed using algorithms such as Mel-frequency cepstral coefficients (MFCCs).

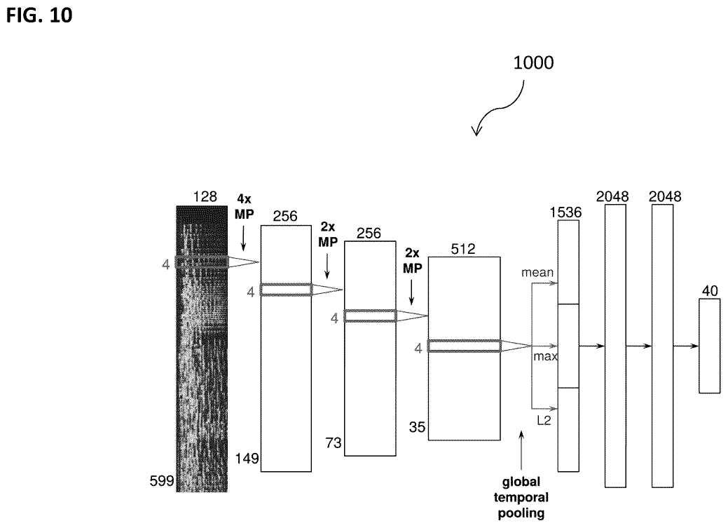

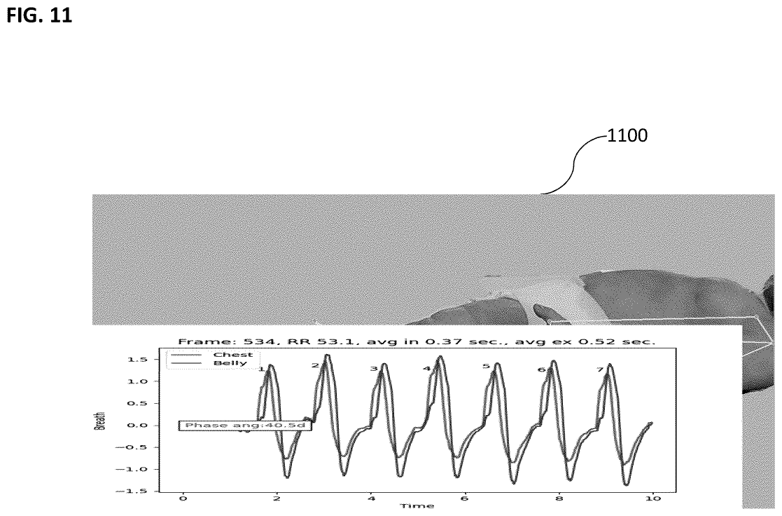

The RNN model is adjusted according to the size of the input layer (receptive field). An example RNN model is depicted in FIG. 10. The example RNN model describes the flow of data and data analysis until the classification of a wheezing sound signature. FIG. 10 depicts an RNN deep learning model architecture 1000, which is an example classification of a sampled window (time-series data) with Asthma attack risk factors. MP stands for Max Pooling convolutional processing.

Pooling is a feature commonly imbibed into Convolutional Neural Network (CNN) architectures. The main idea behind a pooling layer is to “accumulate” features from maps generated by convolving a filter over an image in a way that will be temporal-shift-invariant or space-translation-invariant and will, therefore, capture features while being less sensitive to the location of their maximal response signal. Formally, its function is to progressively reduce the spatial size of the representation to reduce the number of parameters and computations in the network. The most common form of pooling is maximum pooling. Maximum pooling is done in part to help prevent over-fitting by providing an abstracted form of feature representations. Additionally, it reduces the computational cost by reducing the number of parameters to learn, and it provides basic translation invariance to the internal representation. Maximum pooling is done by applying a max filter to (usually) non-overlapping subregions of the initial representation. The other forms of pooling are average and general.

When an exacerbation of a medical condition, such as an Asthma attack, is predicted (based on the tidal breath parameters and wheezing sound signature and supportive environmental Asthma risks factors/triggers), an alert is recorded and generated at block 814. The data alert is recorded locally and remotely at the data center (sent for storage in the data center database within the patient's personal medical records in the patient's user profile/account), at block 816. In addition, an alert message is sent via SMS, WhatsApp™ or any other instant messaging service to the predefined caregiver (one of the parents, family members, kindergarten supervisor, or any other defined caregiver). This parallel messaging and alerting mechanism, ensures that the caregivers will get the alerts on oncoming Asthma (and other) attacks immediately and save valuable time in providing the required preventive treatment to the inspected patient and in particular to children or early-age babies that need medical help and supervision.

FIG. 9 is a flow diagram of a process 900 of captured imagery analysis.

At step 902, the App captures frame-by-frame images from a video or real time smartphone camera streaming.

At step 904, for each frame, the algorithm captures the body points of the chest and the belly.

At step 906, the processing of images from an incorrectly positioned camera is described. The chest key points and/or abdomen key points cannot be identified because the body of the child is not fully visible. In this case, body contour is calculated. Small and isolated closed contours are removed. The upper boundaries of the abdominal and chest walls are assessed using color analysis and contour analysis.

At step 910, the processing of images of a correctly positioned camera is described. The body of a lying child is visible, including the chin, the neck, and the thighs; the upper abdominal wall and upper chest walls are marked by a body-pose artificial neural network (ANN). In this case, the abdominal and chest upper walls are more accurate than in the previous section.

At step 912 an optical flow of the upper abdominal and thoracic wall ascent and descent is calculated. One example of an optical flow algorithm known in the art is the Lucas-Kanade optical flow algorithm.

At step 914 one summation of the optical flow is calculated for the upper chest wall and another for the upper abdominal wall. The summations are of the vertical component of the optical flow within an area that is close to the upper abdominal wall and close to the upper chest wall, respectively. These two summations provide two separate numbers. The summation is a weighted summation, which means that it is weighted with two average values, one around the abdominal wall and one around the chest wall.

In step 916 an exponential filter is calculated for each flow summation. An example of an exponential filter is:

$$abdominal {flow}_{n + 1} = 0.9 * adbominal {flow}_{n} + 0.1 * current adbominal flow chest {flow}_{n + 1} = 0.9 * chest {flow}_{n} + 0.1 * current chest flow$$

In step 918 the filtered abdominal optical flow and the filtered chest optical flow are stored in two separate FIFO buffers, one for the abdomen and one for the chest. In the preferred embodiment, the FIFO buffers hold the last 90 values each.

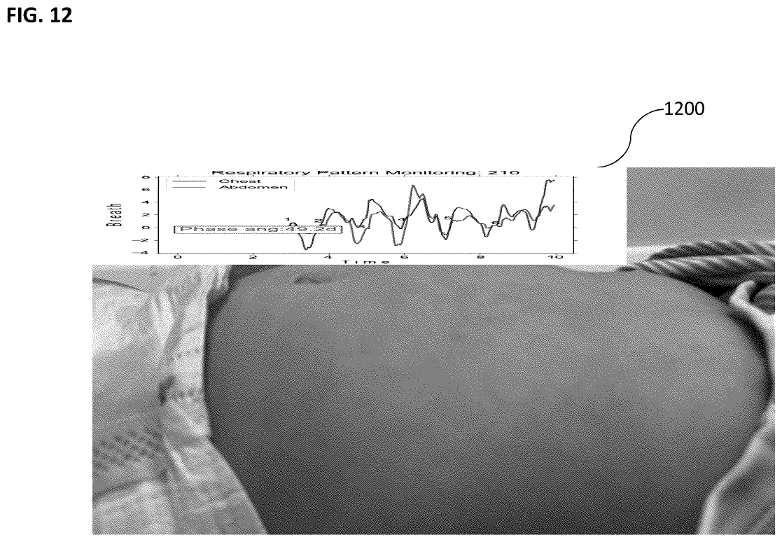

In step 920 the covariance of the two series of 90 values is calculated as a cosine product between two vectors. Arccosine, which is the inverse cosine, is calculated. In healthy children, the angle between the abdomen FIFO and the chest FIFO is less than degrees. If the windpipe is partially obstructed or there are other problems, a phase shift as large as 40 degrees can be formed. This calculated phase shift is displayed on the screen. See FIG. 11 and FIG. 12. If there are not yet 90 values in the buffer, the App goes directly to Step 922.

In step 922 the cycle between the chest wall ascent and descent is calculated. This number is displayed on the screen.

In step 924 a decision is made. If there are more frames, then jump back to step 902 to capture a new frame. If there are no more frames, stop.

FIG. 11 illustrates a calculated phase shift displayed on a screen 1100 in the case of a correctly held smartphone (see FIG. 2(a)). In the figure, the baby presents with a phase shift of about 40 degrees between the abdomen and the chest motion.

FIG. 12 illustrates a calculated phase shift displayed on a screen 1200 in the case of an incorrectly held smartphone (see FIG. 2(c)). (In embodiments, the app has a wizard or tutorial that guides the parents and instructs them how to correctly record the child. In the figure, the baby presents with a phase shift of about 50 degrees between the abdomen and the chest motion. In FIG. 12, the measurement of the phase shift between the abdomen and chest is less accurate than in FIG. 11, but still sufficiently informative about a breathing problem. It reports about 49 degrees when the real value is 50 degrees.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software, by firmware, or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, non-transitory storage media such as a magnetic hard-disk and/or removable media, for storing instructions and/or data. Optionally, a network connection is provided as well. A display and/or a user input device such as a keyboard or mouse are optionally provided as well.

For example, any combination of one or more non-transitory computer-readable (storage) medium(s) may be utilized in accordance with the above-listed embodiments of the present invention. A non-transitory computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: a portable computer diskette, a hard disk, a random-access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing in a standalone computer or in cloud backend server and/or serverless computing. In the context of this document, a computer-readable non-transitory storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electromagnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device.

As will be understood with reference to the paragraphs and the referenced drawings, provided above, various embodiments of computer-implemented methods are provided herein, some of which can be performed by various embodiments of apparatuses and systems described herein and some of which can be performed according to instructions stored in non-transitory computer-readable storage media described herein. Still, some embodiments of computer-implemented methods provided herein can be performed by other apparatuses or systems and can be performed according to instructions stored in computer-readable storage media other than that described herein, as will become apparent to those having skill in the art with reference to the embodiments described herein. Any reference to systems and computer-readable storage media with respect to the following computer-implemented methods is provided for explanatory purposes and is not intended to limit any of such systems and any of such non-transitory computer-readable storage media with regard to embodiments of computer-implemented methods described above. Likewise, any reference to the following computer-implemented methods with respect to systems and computer-readable storage media is provided for explanatory purposes and is not intended to limit any of such computer-implemented methods disclosed herein.

The flowcharts and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As used herein, the singular form “a”, “an” and “the” include plural references unless the context clearly dictates otherwise.

The word “exemplary” is used herein to mean “serving as an example, instance, or illustration”. Any embodiment described as “exemplary” is not necessarily to be construed as preferred or advantageous over other embodiments and/or to exclude the incorporation of features from other embodiments.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

Positional terms such as “upper”, “lower” “right”, “left”, “bottom”, “below”, “lowered”, “low”, “top”, “above”, “elevated”, “high”, “vertical” and “horizontal” as well as grammatical variations thereof as may be used herein do not necessarily indicate that, for example, a “bottom” component is below a “top” component, or that a component that is “below” is indeed “below” another component or that a component that is “above” is indeed “above” another component as such directions, components or both may be flipped, rotated, moved in space, placed in a diagonal orientation or position, placed horizontally or vertically, or similarly modified. Accordingly, it will be appreciated that the terms “bottom”, “below”, “top” and “above” may be used herein for exemplary purposes only, to illustrate the relative positioning or placement of certain components, to indicate a first and a second component or to do both.

“Coupled with” means indirectly or directly “coupled with”.

Where applicable, although state diagrams, flow diagrams or both may be used to describe embodiments, the technique is not limited to those diagrams or to the corresponding descriptions. For example, flow need not move through each illustrated box or state, or in exactly the same order as illustrated and described.

It should be understood that where the claims or specification refer to “a” or “an” element, such reference is not to be construed as there being only one of that element.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Unless otherwise stated, the use of the expression “and/or” between the last two members of a list of options for selection indicates that a selection of one or more of the listed options is appropriate and may be made.

The above-described processes, including portions thereof, can be performed by software, hardware and combinations thereof. These processes and portions thereof can be performed by computers, computer-type devices, workstations, processors, micro-processors, other electronic searching tools, and memory and other non-transitory storage-type devices associated therewith. The processes and portions thereof can also be embodied in programmable non-transitory storage media, for example, compact discs (CDs) or other discs including magnetic, optical, etc., readable by a machine or the like, or other computer usable storage media, including magnetic, optical, or semiconductor storage, or other source of electronic signals.

The processes (methods) and systems, including components thereof, herein have been described with exemplary reference to specific hardware and software. The processes (methods) have been described as exemplary, whereby specific steps and their order can be omitted and/or changed by persons of ordinary skill in the art to reduce these embodiments to practice without undue experimentation. The processes (methods) and systems have been described in a manner sufficient to enable persons of ordinary skill in the art to readily adapt other hardware and software as may be needed to reduce any of the embodiments to practice without undue experimentation and using conventional techniques.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A method of monitoring tidal breath, even in infants, comprising:
passively capturing video imagery of a monitored subject during tidal breathing, wherein an imaging device capturing the video is not in physical contact with the subject body;
analyzing the video imagery to define a region of interest (ROI);
analyzing the ROI to determine Thoraco-Abdominal Asynchrony (TAA).

2. The method of claim 1, wherein the ROI is determined by running at least a portion of the video imagery through a trained machine learning (ML) model.

3. The method of claim 1, wherein the TAA is determined by running at least a portion of the video imagery through a trained machine learning (ML) model.

4. The method of claim 3, wherein the ML model provides a diagnosis based on the video imagery.

5. The method of claim 3, wherein the ML model determines indicators of one or more medical conditions based on the video imagery.

6. The method of claim 3, wherein the video imagery is added to a dataset upon which the ML model is trained.

7. The method of claim 1, further comprising: receiving motion data from motion sensors in physical contact with the monitored subject.

8. The method of claim 7, further comprising: receiving audio data from audio sensors in physical contact with the monitored subject.

9. The method of claim 8, wherein the motion sensors, the audio sensors, or both are disposed on a patch that is adapted to be positioned in physical contact with the monitored subject.

10. The method of claim 1, wherein the video is captured using an integrated camera of a portable computing device (PCD).

11. The method of claim 1, wherein the PCD has a diagnostic program installed thereon, the diagnostic program configured to analyze the captured video.

12. The method of claim 1, wherein the PCD has a communications application installed thereon, wherein the communications application is configured to send or stream the captured video to an off-site computing device, wherein the off-site computing device has a diagnostic program installed thereon, the diagnostic program configured to analyze the captured video.

13. The method of claim 1, further comprising providing an alert when distressed breathing is detected.

14. The method of claim 1, wherein parameters of pulmonary status data are determined by running at least a portion of the video imagery through a trained machine learning (ML) model, the pulmonary status data including: inspiratory time (tI), expiratory time (tE), time to peak tidal expiratory flow/expiratory time (tPTEF/tE), time to peak tidal inspiratory flow/inspiratory time (tPTIF/tI), ratio of inspiratory to expiratory flow at 50% tidal volume (IE50), Relative thoracic contribution, and TAA.

15. The method of claim 14, further comprising providing an alert when monitored pulmonary status data parameters match deterioration patterns identified through the ML model.

16. The method of claim 14, further comprising providing an alert when monitored pulmonary status data parameters show deterioration when compared to previously monitored pulmonary status data parameters for the monitored subject.

17. The method of claim 14, further comprising providing an alert when monitored pulmonary status data parameters indicate asthmatic exacerbation triggers.

18. The method of claim 1, wherein a side view of the monitored subject is imaged by the imaging device.

19. The method of claim 18, wherein the side view is between 0 and 60 degrees normal to a surface on which the monitored subject is resting.

20. The method of claim 18, wherein the side view is between 0 and 20 degrees normal to a surface on which the monitored subject is resting.

21. The method of claim 18, wherein the side view is between 0 and 15 degrees normal to a surface on which the monitored subject is resting.
